(19) United States
(12) Patent Application Publication
(10) Pub. No.: US 2023/0173006 A1
(43) Pub. Date: Jun. 8, 2023

(54) APPLICATION OF TRADITIONAL CHINESE MEDICINE COMPOSITION TO PREPARATION OF MEDICINE FOR TREATING OR PREVENTING CORONAVIRUS INFECTION

(71) Applicant: JIANGSU KANION PHARMACEUTICAL CO., LTD., Lianyungang, Jiangsu (CN)

(72) Inventors: Wei XIAO, Lianyungang (CN); Xinzhuang ZHANG, Lianyungang (CN); Zhipeng KE, Lianyungang (CN); Zhenzhen SU, Lianyungang (CN); Xiaoqian TAO, Lianyungang (CN); Yi DENG, Lianyungang (CN); Zeyu CAO, Lianyungang (CN); Liang CAO, Lianyungang (CN); Zhenzhong WANG, Lianyungang (CN); Xiaolian HE, Lianyungang (CN); Xuehong DONG, Lianyungang (CN); Yanqiu WANG, Lianyungang (CN)

(73) Assignee: JIANGSU KANION PHARMACEUTICAL CO., LTD., Lianyungang, Jiangsu (CN)

(21) Appl. No.: 17/920,163

(22) PCT Filed: Mar. 10, 2021
(86) PCT No.: PCT/CN2021/080023

§ 371 Date: Oct. 20, 2022

(30) Foreign Application Priority Data
Apr. 29, 2020 (CN) 202010356108.X

(51) Int. Cl. A61K 36/13; A61K 36/736; A61K 36/346; A61K 36/23; A61K 36/8966; A61K 36/904; A61K 36/185; A61K 36/484; A61P 31/14

(52) U.S. Cl. CPC A61K 36/13; A61K 36/736; A61K 36/346; A61K 36/23; A61K 36/8966; A61K 36/904; A61K 36/185; A61K 36/484; A61P 31/14

(57) ABSTRACT

An application of a traditional Chinese medicine composition to preparation of a medicine for treating or preventing coronavirus infection. The composition is prepared from the following raw material medicines in parts by weight: 1-3 parts of honey-fried herba ephedrae, 1-10 parts of semen armeniacae amarum, 1-6 parts of radix platycodonis, 1-6 parts of radix peucedani, 1-6 parts of bulbus fritillariae thunbergii, 1-6 parts of radix stemonae, 1-3 parts of oroxylum indicum, 1-10 parts of radix glehniae and 1-3 parts of radix glycyrrhizae. The diluent of the traditional Chinese medicine composition can protect Vero cells infected with 2019-nCov virus.

### TECHNICAL FIELD

The present application relates to the field of medicines for treating diseases, and in particular to application of a traditional Chinese medicine composition to preparation of a medicine for treating or preventing coronavirus infection.

### BACKGROUND

Respiratory tract viral infectious diseases are one of the most common diseases in clinic, with high incidence rate and easy to cause outbreaks among people. Coronavirus is a large virus family, which may cause cold, fever, pneumonia, even Middle East respiratory syndrome (MERS), severe acute respiratory syndrome (SARS) and other serious diseases.

2019-nCoV belongs to a β-genus novel coronavirus, with envelope, round or oval particles, often polymorphism and a diameter of 60-140 nm. The gene characteristics of 2019-nCoV are obviously different from those of SARSr-CoV and MERSr-CoV. The current research shows that the homology of 2019-nCoV with bat SARS-like coronavirus (bat-SL-CoVZC45) reaches more than 85%. Droplet and close contact transmission are the main transmission routes, and aerosol transmission is possible when people is exposed to high-concentration virus aerosol for a long time in a relatively closed environment. The main manifestations of patients are fever, fatigue and dry cough, and a few patients are accompanied by nasal congestion, runny nose and diarrhea. Severe cases have dyspnea after one week. More severe cases rapidly progress to acute respiratory distress syndrome, septic shock, refractory metabolic acidosis, and hemorrhage and coagulation disorders.

The novel coronavirus (COVID-19) epidemic situation seriously threatens the health and life safety of the broad masses of people, and has a significant impact on China's economic development, social stability and foreign exchanges. Due to strong infectivity, rapid transmission and high mortality of 2019-nCoV, there is no effective vaccine or medicine to prevent and treat the virus, so it is particularly urgent to find effective medicines for the virus. Traditional Chinese medicine is the treasure of Chinese medicine culture, and has played an important role in the prevention and control of various epidemic situations. The original medicinal materials of Xingbei Cough Granules related to the present application include: honey-fried herba ephedrae, radix peucedani, bulbus fritillariae thunbergii, radix stemonae, radix platycodonis, radix glehniae, semen armeniacae amarum, oroxylum indicum and radix glycyrrhizae. Indications are: clearing lung qi, relieving cough and reducing sputum. Xingbei Cough Granules are used to treat cough caused by exogenous cold, which is characterized by slight aversion to cold, fever, cough, expectoration, thick and sticky phlegm, dry mouth, bitterness and irritability. Since Xingbei Cough Granules have a good effect of improving the respiratory diseases caused by various viruses and bacteria in clinical application, it is urgent to carry out scientific research on whether Xingbei Cough Granules can effectively prevent and treat coronavirus-related diseases, especially COVID-19.

### SUMMARY

In view of this, the present application aims to provide application of a traditional Chinese medicine composition to preparation of a medicine for treating or preventing coronavirus infection, wherein the traditional Chinese medicine composition includes: 1-3 parts of honey-fried herba ephedrae, 1-10 parts of semen armeniacae amarum, 1-6 parts of radix platycodonis, 1-6 parts of radix peucedani, 1-6 parts of bulbus fritillariae thunbergii, 1-6 parts of radix stemonae, 1-3 parts of oroxylum indicum, 1-10 parts of radix glehniae and 1-3 parts of radix glycyrrhizae.

Further, diseases infected with coronavirus include COVID-19 (novel coronavirus pneumonia).

Further, the traditional Chinese medicine composition includes: 1-3 parts of honey-fried herba ephedrae, 1-10 parts of semen armeniacae amarum, 1-6 parts of radix platycodonis, 1-6 parts of radix peucedani, 1-6 parts of bulbus fritillariae thunbergii, 1-6 parts of radix stemonae, 1-3 parts of oroxylum indicum, 1-10 parts of radix glehniae and 1-3 parts of radix glycyrrhizae.

Further, the traditional Chinese medicine composition includes:1-1.5 parts of honey-fried herba ephedrae, 2-4 parts of semen armeniacae amarum, 1-3 parts of radix platycodonis, 1-3 parts of radix peucedani, 1-3 parts of bulbus fritillariae thunbergii, 1-3 parts of radix stemonae,1-1.5 parts of oroxylum indicum, 2-4 parts of radix glehniae and 1-1.5 parts of radix glycyrrhizae.

Preferably, the traditional Chinese medicine composition includes: 1 part of honey-fried herba ephedrae, 3.33 parts of semen armeniacae amarum, 2 parts of radix platycodonis, 2 parts of radix peucedani, 2 parts of bulbus fritillariae thunbergii, 2 parts of radix stemonae, 1 part of oroxylum indicum, 3.33 parts of radix glehniae and 1 part of radix glycyrrhizae.

Optionally, the traditional Chinese medicine composition includes: 100 g of honey-fried herba ephedrae, 333 g of semen armeniacae amarum, 200 g of radix platycodonis, 200 g of radix peucedani, 200 g of bulbus fritillariae thunbergii, 200 g of radix stemonae, 100 g of oroxylum indicum, 333 g of radix glehniae and 100 g of radix glycyrrhizae.

Specifically, the medicine for treating or preventing coronavirus infection includes an oral administration dosage form, an injection administration dosage form or an external administration dosage form.

Specifically, the medicine for treating or preventing coronavirus infection includes but is not limited to decoction, tablet, capsule, granule, pill, injection, condensed decoction, suspending agent, dispersing agent, syrup, suppository, gel, aerosol, patch and oral liquid.

The raw material medicines used in the present application may be directly ground into powder, or may be extracts prepared by the conventional method in the field; and in the composition, several components may be directly ground into powder according to a traditional method, or extracts may be prepared by a conventional method, thereby being prepared into various dosage forms, achieving better curative effect and being more conducive to being prepared into modern medicine dosage forms.

Preferably, a preparation method of the traditional Chinese medicine composition includes the following steps: taking the honey-fried herba ephedrae, the radix peucedani, the bulbus fritillariae thunbergii and the radix stemonae, adding 50%-80% ethanol in an amount which is 4-8 times the weight of the honey-fried herba ephedrae, the radix peucedani, the bulbus fritillariae thunbergii and the radix stemonae, refluxing for 1-3 hours, filtering, and concentrating the filtrate under reduced pressure to obtain a clear paste; heating and boiling the radix platycodonis, the radix glehniae, the oroxylum indicum and the radix glycyrrhizae with water, adding the semen armeniacae amarum and boiling, filtering, and concentrating the filtrate under reduced pressure to obtain a clear paste; and combining the two clear pastes to obtain the traditional Chinese medicine composition.

Further, the preparation method of the traditional Chinese medicine composition may include the following steps: adding 70% ethanol, in an amount which is 6 times the weight of the herba ephedrae, the radix peucedani, the bulbus fritillariae thunbergii and the radix stemonae, into the herba ephedrae, the radix peucedani, the bulbus fritillariae thunbergii and the radix stemonae, refluxing for 2 hours, filtering, adding 70% ethanol, in an amount which is 4 times the weight of medicine residues, into the medicine residues, refluxing for 2 hours, filtering, combining the filtrates, concentrating by using a thin film under reduced pressure to a relative density of 1.07 to obtain an alcohol-extracted clear paste; adding water, in an amount which is 10 times the weight of the semen armeniacae amarum, the radix glehniae, the radix platycodonis, the radix glycyrrhizae and the oroxylum indicum, into the semen armeniacae amarum, the radix glehniae, the radix platycodonis, the radix glycyrrhizae and the oroxylum indicum, adding the semen armeniacae amarum after boiling, boiling for 1.5 hours, filtering, adding water, in an amount which is 8 times the weight of medicine residues, into the medicine residues, boiling for 1.5 hours, filtering, combining the filtrate, concentrating by using a thin film under reduced pressure to a relative density of 1.09 to obtain a water-extracted clear paste; and combining, uniformly mixing and drying the clear pastes.

The present application further provides a preparation method of the above granules. Optionally, the granules are prepared by uniformly mixing spray-dried powder prepared by the preparation method of any one of the above compositions, 37%-41% dextrin and 1% aspartame, and performing dry granulation.

The granules of the traditional Chinese medicine composition provided by the present application are prepared into 1.7664 g of crude medicine/mL, which can show a good protective effect on VeroE6 cells infected with 2019-nCoV virus and can inhibit the replication of 2019-nCoV in the cells at 1:100, 1:1000 and 1:10000 times dilution concentrations, so that the infected cells do not have cytopathic effect, indicating that the traditional Chinese medicine composition provided by the present application has a good effect of treating or preventing coronavirus-related diseases.

### DETAILED DESCRIPTION OF EMBODIMENTS

The present application provides application of a traditional Chinese medicine composition. Those skilled in the art may learn from the contents of the specification to appropriately improve process parameters. It should be noted that all the similar substitutions and modifications are obvious to those skilled in the art and should be considered to be included in the present application. The method and application of the present application have been described by preferred embodiments, and relevant personnel obviously can modify or appropriately change and combine the method and application of the specification without departing from the content, spirit and scope of the present application to implement and apply the technology of the present application.

Unless otherwise specified, medicines, biomaterials or instruments used in the present application are all common commercial products and can be purchased in the market.

The present application will be further described below with reference to embodiments.

Embodiment 1 Preparation of traditional Chinese medicine compound granules

The raw materials include: 100 g of honey-fried herba ephedrae, 333 g of semen armeniacae amarum, 200 g of radix platycodonis, 200 g of radix peucedani, 200 g of bulbus fritillariae thunbergii, 200 g of radix stemonae, 100 g of oroxylum indicum, 333 g of radix glehniae and 100 g of radix glycyrrhizae.

70% ethanol, in an amount which was 6 times the weight of medicinal materials (decoction pieces) (herba ephedrae, radix peucedani, bulbus fritillariae thunbergii and radix stemonae), was added in the medicinal materials (decoction pieces) herba ephedrae, radix peucedani, bulbus fritillariae thunbergii and radix stemonae, refluxing was performed for 2 hours, filtering was performed, 70% ethanol, in an amount which was 4 times the medicinal residues, refluxing was performed for 2 hours, filtering was performed, the filtrates were combined, the filtrates was thin film concentrated under reduced pressure to a relative density of 1.07 (60° C.) to obtain an alcohol-extracted clear paste, and the clear paste was placed for future use. Water, in an amount which was 10 times the weight of five medicines (decoction pieces), was added into the five medicines (decoction pieces) such as semen armeniacae amarum, radix glehniae, radix platycodonis, radix glycyrrhizae and oroxylum indicum, where the semen armeniacae amarum was added after boiling, boiling was performed for 1.5 hours, filtering was performed, water, in an amount which was 8 times the weight of medicinal residues, was added in the medicinal residues, boiling was performed for 1.5 hours, filtering was performed, the filtrates were combined, and the filtrates was thin film concentrated under reduced pressure until a relative density of 1.09 (60° C.) to obtain a water-extracted clear paste. The clear paste was combined, mixed uniformly and spray-dried to obtain dry powder. Dextrin (37%-41% of the theoretical granule yield) and aspartame (1% of the theoretical granule yield) were added into the dry powder and mixed uniformly, dry granulation was performed, and subpackaging was performed by using aluminum foils (4.0 g in each bag) to obtain 1000 g of XingBei Cough granules.

### Pharmaceutical Effect Test Example

1. Experimental materials

Test medicine: 4 g of medicine prepared in Embodiment 1 of the present application was added with 10 ml of cell maintaining liquid. That is the original concentration 1 (1.7664 g crude medicine/mL).

Vero cell: preserved in Cell Bank of Institute of Microbiological Epidemiology.

2019-nCoV virus: having a titer of 10⁷ TCID₅₀/ml, and preserved in Virus Reservoir of Institute of Microbiological Epidemiology and P3 virus seed refrigerator at −80° C. The virus titer was 100TCID₅₀.

2. Experimental method

100 μl of Vero cells with the concentration of 1×10⁵cell/ml was added into each well of a sterile 96-well culture plate, and cultivation was performed under the conditions of 37° C. and 5% CO₂ for 24 hours; the test medicine was diluted into 6 concentrations (1:1, 1:10, 1:100, 1:1000, 1:10000, 1:100000), 4 duplicate-wells were set for each concentration, 100 μl of the test medicine was added in each well, action was performed for 1 hour, then isovolumetric 100 TCID50 viruses were added into each well and action was performed for 1 hour; after 1 hour, all cell culture fluid in the 96-well culture plate was discarded, and the diluted medicine liquid was added; meanwhile, cell control, blank control (solvent control) and virus control (negative control) were set; cells were incubated in an incubator under the conditions of 37° C. and 5% CO₂ for 3 days; cytopathic effect (CPE) was observed under an optical microscope, complete cytopathic effect was recorded as “++++”, 75% cytopathic effect was recorded as “+++”, 50% cytopathic effect was recorded as “++”, 25% cytopathic effect was recorded as “+”, and non-cytopathic effect was recorded as “-”.

3. Experiment condition: all the experimental operations were completed in BSL-3 laboratory.

4. Result judgment: the cell without CPE has the effective concentration for inhibiting virus, and the cell with CPE has the non-effective concentration.

5. Experimental results:

According to the present application, the medicine group has 6 concentrations, cytotoxicity is present at high concentrations (1:1 and 1:10), 2019-nCoV replication can be effectively inhibited at the concentrations of 1:100, 1:1000 and 1:10000, and there is no CPE activity caused by virus inhibition at low concentration (1:100000). The results are shown in Table 1.

TABLE 1
Anti-2019-nCoV Effect of Medicine

| Name of Test Medicine | Concentration | Result |
|---|---|---|
| Medicines of the | 1:1 | / |
| present application | 1:10 | / |
| | 1:100 | − |
| | 1:1000 | + |
| | 1:10000 | ++ |
| | 1:100000 | ++++ |
| Virus control | | ++++ |
| Cell control | | − |

/ Cytotoxicity, “−” means no cytopathic effect; and “++++” means complete cytopathic effect.

6. Conclusion:

According to the screening results of cell level, the samples prepared according to Embodiment 1 of the present application can inhibit CPE effect caused by virus at the concentrations of 1:100, 1:1000 and 1:10000, where the sample at the concentration of 1:100 has the strongest effect of inhibiting the CPE effect caused by virus. There is cytotoxicity at high concentrations (1:1 and 1:10), and there is no visible CPE activity caused by virus at low concentration (1:100000), indicating that the medicine provided by the present application has good anti-2019-nCoV activity in vitro.

It should be noted that those skilled in the art may learn from the contents of the specification to appropriately improve process parameters, and all the similar substitutions and modifications are obvious to those skilled in the art and should be considered to be included in the present application.

1-9. (canceled)

10. Application of a traditional Chinese medicine composition to preparation of a medicine for treating or preventing coronavirus infection, wherein the Chinese medicine composition comprising: 1-3 parts of honey-fried herba ephedrae, 1-10 parts of semen armeniacae amarum, 1-6 parts of radix platycodonis, 1-6 parts of radix peucedani, 1-6 parts of bulbus fritillariae thunbergii, 1-6 parts of radix stemonae, 1-3 parts of oroxylum indicum, 1-10 parts of radix glehniae and 1-3 parts of radix glycyrrhizae.

11. The application according to claim 10, wherein the coronavirus infection comprises COVID-19 infection.

12. The application according to claim 10, wherein the traditional Chinese medicine composition comprises: 1-1.5 parts of honey-fried herba ephedrae, 2-4 parts of semen armeniacae amarum, 1-3 parts of radix platycodonis, 1-3 parts of radix peucedani, 1-3 parts of bulbus fritillariae thunbergii, 1-3 parts of radix stemonae, 1-1.5 parts of oroxylum indicum, 2-4 parts of radix glehniae and 1-1.5 parts of radix glycyrrhizae.

13. The application according to claim 10, wherein the traditional Chinese medicine composition comprises: 1 part of honey-fried herba ephedrae, 3.33 parts of semen armeniacae amarum, 2 parts of radix platycodonis, 2 parts of radix peucedani, 2 parts of bulbus fritillariae thunbergii, 2 parts of radix stemonae, 1 part of oroxylum indicum, 3.33 parts of radix glehniae and 1 part of radix glycyrrhizae.

14. The application according to claim 10, wherein the traditional Chinese medicine composition comprises: 100 g of honey-fried herba ephedrae, 333 g of semen armeniacae amarum, 200 g of radix platycodonis, 200 g of radix peucedani, 200 g of bulbus fritillariae thunbergii, 200 g of radix stemonae, 100 g of oroxylum indicum, 333 g of radix glehniae and 100 g of radix glycyrrhizae.

15. The application according to claim 10, wherein the medicine for treating or preventing coronavirus infection comprises an oral administration dosage form, an injection administration dosage form or an external administration dosage form.

16. The application according to claim 15, wherein the medicine for treating or preventing coronavirus infection comprises decoction, tablet, capsule, granule, pill, injection, condensed decoction, suspending agent, dispersing agent, syrup, suppository, gel, aerosol, patch and oral liquid.

17. The application according to claim 10, wherein a preparation method of the traditional Chinese medicine composition comprises the following steps: taking the honey-fried herba ephedrae, the radix peucedani, the bulbus fritillariae thunbergii and the radix stemonae, adding 50%-80% ethanol in an amount which is 4-8 times the weight of the honey-fried herba ephedrae, the radix peucedani, the bulbus fritillariae thunbergii and the radix stemonae, refluxing for 1-3 hours, filtering, concentrating the filtrate under reduced pressure to obtain a clear paste; heating and boiling the radix platycodonis, the radix glehniae, the oroxylum indicum and the radix glycyrrhizae with water, then adding the semen armeniacae amarum and boiling, filtering, and concentrating the filtrate under reduced pressure to obtain a clear paste; and combining, uniformly mixing and drying the two clear pastes.

18. The application according to claim 17, wherein the preparation method of the traditional Chinese medicine composition comprises the following steps: adding 70% ethanol, in an amount which is 6 times the weight of the herba ephedrae, the radix peucedani, the bulbus fritillariae thunbergii and the radix stemonae, into the herba ephedrae, the radix peucedani, the bulbus fritillariae thunbergii and the radix stemonae, refluxing for 2 hours, filtering, adding 70% ethanol, in an amount which is 4 times the weight of medicine residues, into the medicine residues, refluxing for 2 hours, filtering, combining the filtrates, concentrating by using a thin film under reduced pressure to a relative density of 1.07 to obtain an alcohol-extracted clear paste; adding water, in an amount which is 10 times the weight of the semen armeniacae amarum, the radix glehniae, the radix platycodonis, the radix glycyrrhizae and the oroxylum indicum, into the semen armeniacae amarum, the radix glehniae, the radix platycodonis, the radix glycyrrhizae and the oroxylum indicum, adding the semen armeniacae amarum after boiling, boiling for 1.5 hours, filtering, adding water, in an amount which is 8 times the weight of medicine residues, into the medicine residues, boiling for 1.5 hours, filtering, combining the filtrate, concentrating by using a thin film under reduced pressure to a relative density of 1.09 to obtain a water-extracted clear paste; and combining, uniformly mixing and drying the clear pastes.
